(19) United States
(12) Patent Application Publication
(10) Pub. No.: US 2012/0258117 A1
(43) Pub. Date: Oct. 11, 2012

(54) Methods and compositions for modulation of histone ubiquitination

(76) Inventors: C. W. Xu, Pasadena, CA (US); Zhen Gao, St. Louis, MO (US)

(21) Appl. No.: 13/440,510

(22) Filed: Apr. 5, 2012

Related U.S. Application Data
(60) Provisional application No. 61/472,868, filed on Apr. 7, 2011.

(51) Int. Cl. A61K 39/395; A61P 35/00; A61K 31/7088; C07H 21/02; A61K 31/05; C07K 16/40; C07C 39/21; C07H 21/04; C12N 5/09; A61K 31/713

(52) U.S. Cl. 424/158.1; 435/375; 514/44 A; 514/734; 530/389.7; 568/729; 536/23.1; 536/24.5; 514/44 R

(57) ABSTRACT

Provided are methods and compositions for inhibiting mono-ubiquitination of a histone in a cell. Also provided are methods and compositions for inducing senescence in a cell. Additionally provided are methods and compositions for treatment of cancer, including glioma. In particular, these methods and compositions may include an inhibitor of a ubiquitin ligase.

### BACKGROUND OF THE INVENTION

Histone modifications are intimately involved in transcriptional regulation. Among histone acetylation, methylation, phosphorylation and mono-ubiquitination, the role of mono-ubiquitination of histones is least understood, despite the fact that histone H2A is the first protein shown to be covalently conjugated with ubiquitin (M. A. Osley, Brief Funct Genomic Proteomic 5 (2006) 179-89). In budding yeast, H2B is the only histone that is mono-ubiquitinated at K123 (uH2B), which constitutes approximately 15% of the total H2B during exponential phase, but is not detectable in stationary phase (L. Dong, and C. W. Xu, J Biol Chem 279 (2004) 1577-80). Bre1, a Rad6-associated RING finger protein, is the E3 ubiquitin ligase of H2B in yeast. RNF20, a human homolog of yeast Bre1, has been identified, suggesting that mono-ubiquitination of histone H2B is conserved from yeast to humans (W. W. Hwang, et al., Mol Cell 11 (2003) 261-6). Using mono-ubiquitination of histone H2B at K123 (uH2B) in yeast as a model, it has been shown that glucose induces uH2B through glycolysis, revealing a paradigm of nutritional regulation of histone modifications. It has been also demonstrated that glycolysis is also required for mono-ubquitination of histone H2B at K120, the orthologous site of K123 of yeast histone H2B, in both human primary and tumor cells (Z. Gao, and C. W. Xu, Biochem Biophys Res Commun 404 (2011) 428-33

Resveratrol (3,4′,5-trihydroxy-trans-stilbene) is a polyphenol naturally occurring in grapes and other plants. Although resveratrol modulates multiple pathways in tumor cells, how resveratrol or its affected pathways converge on chromatin to mediate its effects is not known. Resveratrol, which is synthesized de novo in food and medicinal plants, presumably in response to stress or infection, has been shown to inhibit proliferation in a variety of tumor cells such as skin, breast, prostate, and lung through multiple pathways.

### BRIEF SUMMARY OF THE INVENTION

In one aspect, the present invention provides a method of inducing senescence in a cell comprising contacting the cell with an inhibitor of mono-ubiquitination of a histone.

In another aspect, the present invention provides a method of inhibiting mono-ubiquitination of a histone in a cell comprising contacting the cell with a negative modulator of a ubiquitin ligase.

In another aspect, the present invention provides a method of inhibiting mono-ubiquitination of a histone in a cell comprising contacting the cell with a positive modulator of a ubiquitin-specific protease.

Another aspect of the invention provides a method of treatment for cancer comprising administration to a subject in need of treatment a composition comprising a negative modulator of a ubiquitin ligase.

Another aspect of the invention provides a method of treatment for cancer comprising administration to a subject in need of treatment a composition comprising a positive modulator of a ubiquitin-specific protease.

In another aspect, the present invention provides a method of treatment for cancer comprising administration to a subject in need of treatment a composition comprising a negative modulator of RNF20.

In another aspect, the present invention provides a method of treatment for glioma comprising administration to a subject in need of treatment a composition comprising an effective amount of resveratrol.

In a further aspect, the present invention provides a composition comprising at least one negative modulator of a ubiquitin ligase.

### BRIEF DESCRIPTION OF THE DRAWINGS

FIGS. 1A-C demonstrate resveratrol inhibited cell proliferation and induced cellular senescence of glioma cells. (A) 5×10⁴ U87 or U118 cells were seeded in 60 mm dishes and were treated with resveratrol (RV), acetone, or mock-treated. Viable (Trypan blue excluded) cells were counted on D3, D6, and D9. The means and standard deviations were derived from cell count on three dishes. (B) Expression of senescence-associated-β-galactosidase (SA-β-gal) in resveratrol-treated U87 glioma cells is shown. A resveratrol-induced senescent cell is highlighted. (C) Expression of senescence-associated-β-galactosidase (SA-β-gal) in U118 glioma cells is shown. A resveratrol-induced senescent cell is highlighted.

FIGS. 2A and B are graphical representations of resveratrol inhibited clonogenic efficiency of human glioma cells in vitro. (A) Resveratrol-treated U87 and U118 (9-day treatment, 1000 to 4000 cells) were re-plated. After 14 days, the cultured cells were stained with 0.4% Coomassie Blue G250 in 50% methanol and 5% acetic acid. The graph shows the cell plating efficiencies (PE), which were calculated using the formula: PE=(number of colonies counted/number of cells seeded)×100%. (B) Tumor growth volume from subcutaneously-injected U87 and U118 is depicted. U87 tumor volume was measured at 27 days post injection while U118 tumor volume at 125 days post injection. Five mice were used for each treatment.

FIGS. 3A and B demonstrate resveratrol inhibited mono-ubiquitination of histone H2B at K120 (uH2B) in glioma cells. (A) Chronic treatment (up to 9-day) with lower doses of resveratrol inhibited uH2B of U87. (B) Chronic treatment (up to 9-day) with lower doses of resveratrol inhibited uH2B of U118.

FIGS. 4A and B demonstrate depletion of RNF20 inhibited mono-ubiquitination of histone H2B at K120 (UH2B) and induced cellular senescence of human glioma cells. (A) Infection of lentivirus shRNF20 inhibited expression of RNF20 and uH2B. Total cellular proteins were prepared from U87 and U118 infected with either shLuc (MOI=10) or shRNF20 (MOI=10). Equal amounts of proteins were separated by SDS-PAGE and immunoblotted for RNF20, uH2B, and H2B. (B) U87 and U118 cells were infected with either shLuc (MOI=10) or shRNF20 (MOI=10). On Day 6 post-infection, the cells were analyzed for expression of SA-β-gal. Two senescent cells are highlighted.

FIG. 5 shows the results of acute (4-hour) treatment with resveratrol inhibited uH2B in a variety of tumor and non-malignant cells. U87 and U118, human glioma cells; MCF7, human breast cancer cells; H1299, non-small cell lung carcinoma cells; PC3, human prostate cancer cells; HDF, non-malignant normal human dermal fibroblasts; and HMEpC, normal human mammary epithelial cells.

### DETAILED DESCRIPTION OF THE INVENTION

Resveratrol has a variety of activities including anti-cancer, anti-inflammatory, anti-angiogenic, and chemosensitizing properties in vitro and in vivo models by targeting multiple cellular targets. In accordance with the present invention it has been demonstrated that resveratrol modulates histone modifications.

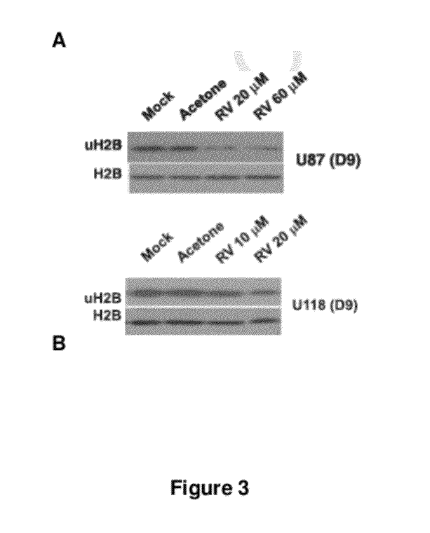

Mono-ubiquitination (uH2B) occurs in a rapidly-dividing (exponential-phase) yeast but is not detectable in stationary phase (G0) yeast. In accordance with the present invention, it has been demonstrated that global uH2B levels in proliferating tumor cells are much higher than those in resveratrol-induced senescent tumor cells (FIG. 3). Further, it has been discovered that depletion of RNF20 induces flat and enlarged senescent glioma cells.

Evidence indicates that therapy-induced cellular senescence correlates with better clinical outcome than therapy-induced apoptosis (I. B. Roninson, Cancer Res 63 (2003) 2705-15). Cellular senescence was first noted in the 1960s as a permanent arrest of cell cycle of non-malignant cells, which undergo limited number of cell divisions before entering cellular senescence, a permanent and irreversible proliferation arrest. The bypassing of cellular senescence is believed to be a step in tumorigenesis (G. P. Dimri, Cancer Cell 7 (2005) 505-12). Resveratrol-induced cellular senescence in human glioma cells as discovered herein provides a novel therapy for glioma, which currently lacks an effective treatment. Further, it has been discovered in accordance with the present invention that depletion of RNF20 inhibits uH2B and induces cellular senescence of malignant human glioma cells, and thereby recapitulates the effects of resveratrol treatment. The present thus further provides methods of inhibition of ubiquitination as a novel therapy for glioma.

It has been discovered herein that resveratrol inhibits cell proliferation and induces cellular hypertrophy in glioma cells by transforming spindle-shaped cells to enlarged, irregular and flattened-shaped ones. Additionally, resveratrol-induced hypertrophic cells express senescence-associated-β-galactosidase, indicating that resveratrol induces cellular senescence in glioma cells. In addition, it has been demonstrated herein that resveratrol inhibits clonogenic efficiencies and tumor growth in a xenograft model. Furthermore, it has been found herein that acute treatment with resveratrol inhibits mono-ubiquitination of histone H2B at K120 (uH2B) in breast, prostate, pancreatic, lung, brain tumor cells as well as primary human cells. Chronic treatment with low doses of resveratrol also inhibits uH2B in the resveratrol-induced senescent glioma cells. Moreover, depletion of RNF20, a ubiquitin ligase of histone H2B, inhibits uH2B and induces cellular senescence in glioma cells, thereby recapitulating the effects of resveratrol. While not intending to be bound by any theory of operation, the results indicate that uH2B is a novel direct or indirect chromatin target of resveratrol and that RNF20 plays a role in inhibiting cellular senescence programs that are intact in glioma cells.

In certain embodiments of the invention, methods are provided for inhibiting mono-ubiquitination of a histone.

Particular embodiments of the invention provide methods of inducing senescence in a cell. According to certain embodiments, a cell may be contacted with a composition comprising an inhibitor of mono-ubiquitination of a histone. In a preferred embodiment, the histone for which mono-ubiquitination is inhibited is H2B. In some embodiments, the inhibitor of mono-ubiquitination of the histone comprises a negative modulator of a ubiquitin ligase. In certain embodiments, a ubiquitin ligase that is inhibited is at least one of RNF20, RNF40, HR6A/B and UbcH6. In a preferred embodiment, the ubiquitin ligase in RNF20. Negative modulators of PKM2, PAF1 and RTF1 may also be used in the present methods. In a preferred embodiment, the composition comprises resveratrol.

The methods and compositions of the present invention which use or comprise resveratrol may also use or comprise structural and functional analogs of resveratrol that maintain the ability of resveratrol to induce senescence in a cell.

In some embodiments, the inhibitor of mono-ubiquitination of the histone comprises a positive modulator of a ubiquitin-specific protease. In certain embodiments, the ubiquitin-specific protease that is positively modulated is at least one of USP22 and USP7/HAUSP.

In some embodiments, the cell is a cancer cell. Exemplary types of cancer cells include, but are not limited to, breast, prostate, pancreatic, lung, and brain tumor cells. In a preferred embodiment, the cell is a glioma cell.

Another embodiment provides methods of treatment for a cancer comprising administration to a subject in need of treatment an effective amount of a composition comprising an inhibitor of mono-ubiquitination of a histone. In certain embodiments, the histone is H2B. In a preferred embodiment, treatment comprises administration to a subject in need of treatment an effective amount of a composition comprising a negative modulator of a ubiquitin ligase in an amount effective to inhibit mono-ubiquitination of a histone in cancer cells in the subject. In certain embodiments, a ubiquitin ligase that is inhibited is at least one of RNF20, RNF40, HR6A/B and UbcH6. In a preferred embodiment, the ubiquitin ligase inhibited by treatment is RNF20. Negative modulators of PKM2, PAF1 and RTF1 may also be used in the present methods. In a preferred embodiment, the composition comprises resveratrol.

In some embodiments, treatment comprises administration to a subject in need of treatment an effective amount of a composition comprising a positive modulator of a ubiquitin-specific protease in an amount effective to inhibit mono-ubiquitination of a histone in cancer cells in the subject. In certain embodiments, the ubiquitin-specific protease that is positively modulated is at least one of USP22 and USP7/HAUSP. In certain embodiments, the cancer is glioma.

The therapeutic agents of the present invention may be used alone or in combination with other cancer therapies including, for example, chemotherapy, radiation therapy, immunotherapy and gene therapy.

In another embodiment, the method of treatment further comprises the administration of a second therapeutic agent. In a preferred embodiment, the second therapeutic agent is an anticancer agent. The second agent may be administered before, after, or concurrently with the negative modulator of ubiquitin ligase.

A further embodiment provides compositions effective for the inhibition of mono-ubiquitination of a histone. Such compositions may be used in connection with various methods according to aspects of the invention. In a preferred embodiment, the composition inhibits the mono-ubiquitination of histone H2B. Preferably, the composition comprises at least one negative modulator of a ubiquitin ligase or at keast one positive modulator of a ubiquitin-specific protease. In certain preferred embodiments, compositions are provided that inhibit the ubiquitin ligase RNF20.

In a preferred embodiment, the composition comprises resveratrol (3,4′,5-trihydroxy-trans-stilbene).

Positive modulators of ubiquitin-specific protease include USP22, USP7/HAUSP and biologically active fragments thereof, nucleic acids encoding USP22, USP7/HAUSP and biologically active fragments thereof, and vectors comprising the nucleic acids.

Negative modulators of ubiquitin ligase include, but are not limited to: antibodies, aptamers, antisense oligonucleotides, interfering RNA, and small molecule inhibitors. These agents are suitable for various methods described herein.

Exemplary ubiquitin ligase inhibitors include, but are not limited to:
a) antibodies that immunoreact (bind) with a ubiquitin ligase (also known as ubiquitin ligase antibodies or anti-ubiquitin ligase antibodies);
(b) fragments of (a) that retain antigen binding activity;
(c) polypeptides that comprise an antigen binding domain of (a) or (b) and that bind said antigen;
(d) antisense oligonucleotides that inhibit ubiquitin ligase transcription or translation;
(e) aptamers that inhibit a ubiquitin ligase;
(f) short interfering RNAs (siRNA, RNAi) that inhibit ubiquitin ligase translation;
(g) small molecule inhibitors of ubiquitin ligase; and
(f) combinations thereof.

In a preferred embodiment, the small molecule inhibitor of ubiquitin ligase is resveratrol. In another preferred embodiment, the inhibitor of ubiquitin ligase is a siRNA.

### Antibodies

Anti-ubiquitin ligase antibodies, including, for example, monoclonal, polyclonal, human, humanized and bispecific antibodies may be used in the methods described herein. Polyclonal or monoclonal therapeutic anti-ubiquitin ligase antibodies useful in practicing this invention may be prepared in laboratory animals or by recombinant DNA techniques using the methods known in the art, or may be obtained commercially Polyclonal antibodies to a ubiquitin ligase molecule or a fragment thereof containing the target amino acid sequence generally are raised in animals by multiple subcutaneous (sc) or intraperitoneal (ip) injections of the ubiquitin ligase molecule in combination with an adjuvant such as Freund's adjuvant (complete or incomplete). To enhance immunogenicity, it may be useful to first conjugate the ubiquitin ligase molecule or a fragment containing the target amino acid sequence of to a protein that is immunogenic in the species to be immunized, e.g., keyhole limpet hemocyanin, serum albumin, bovine thyroglobulin, or soybean trypsin inhibitor using a bifunctional or derivatizing agent, for example, maleimidobenzoyl sulfosuccinimide ester (conjugation through cysteine residues), N-hydroxysuccinimide (through lysine residues), glutaraldehyde, succinic anhydride, SOC₁, or R¹N═C═NR, where R and R¹ are different alkyl groups. Alternatively, ubiquitin ligase-immunogenic conjugates can be produced recombinantly as fusion proteins.

Animals may be immunized against the immunogenic ubiquitin ligase conjugates or derivatives (such as a fragment containing the target amino acid sequence) by combining about 1 mg or about 1 microgram of conjugate (for rabbits or mice, respectively) with about 3 volumes of Freund's complete adjuvant and injecting the solution intradermally at multiple sites. Approximately 7 to 14 days later, animals are bled and the serum is assayed for anti-ubiquitin ligase titer. Animals are boosted with antigen repeatedly until the titer plateaus. Preferably, the animal is boosted with the same ubiquitin ligase molecule or fragment thereof as was used for the initial immunization, but conjugated to a different protein and/or through a different cross-linking agent. In addition, aggregating agents such as alum may be used in the injections to enhance the immune response.

Monoclonal antibodies may be prepared by recovering spleen cells from immunized animals and immortalizing the cells in conventional fashion, e.g. by fusion with myeloma cells. The clones are then screened for those expressing the desired antibody.

Preparation of antibodies using recombinant DNA methods, such as the phagemid display method, may be accomplished by methods known in the art and may be performed using commercially available kits, as for example, the Recombinant Phagemid Antibody System available from Pharmacia (Uppsala, Sweden), or the SurfZAP™. phage display system (Stratagene Inc., La Jolla, Calif.). Human antibodies may also be prepared with yeast display methods as disclosed, for example, by E. T. Boder and K. D. Wittrup, Nat Biotech (1997) 553-557.

Preferably, antibodies for administration to humans will be “humanized”, or chimeric, i.e. made to be compatible with the human immune system such that a human patient will not develop an immune response to the antibody. Even more preferably, human antibodies prepared using methods such as those described for example, in Lonberg, et al., Nature Genetics, 7:13-21 (1994) are preferred for therapeutic administration to patients. Further methods for making antibodies are disclosed in U.S. Patent Application Publication 20110076761.

The term “antigen binding domain” or “antigen binding region” refers to that portion of the selective binding agent (such as an antibody molecule) which contains the specific binding agent amino acid residues that interact with an antigen and confer on the binding agent its specificity and affinity for the antigen. In an antibody, the antigen binding domain is commonly referred to as the “complementarity determining region”, or “CDR.”

### Aptamers

Recent advances in the field of combinatorial sciences have identified short polymer sequences with high affinity and specificity to a given target. For example, SELEX technology has been used to identify DNA and RNA aptamers with binding properties that rival mammalian antibodies. The field of immunology has generated and isolated antibodies or antibody fragments which bind to a myriad of compounds and phage display has been utilized to discover new peptide sequences with very favorable binding properties. Based on the success of these molecular evolution techniques, ligands can be created which bind to a ubiquitin ligase. In each case, a loop structure is often involved with providing the desired binding attributes as in the case of: aptamers which often utilize hairpin loops created from short regions without complimentary base pairing, naturally derived antibodies that utilize combinatorial arrangement of looped hyper-variable regions and new phage display libraries utilizing cyclic peptides that have shown improved results when compare to linear peptide phage display results. Thus, high affinity ligands can be created and identified by combinatorial molecular evolution techniques. For the present invention, molecular evolution techniques can be used to isolate ligands specific for ubiquitin ligases. For more on aptamers, see generally, Gold, L., Singer, B., He, Y. Y., Brody. E., “Aptamers As Therapeutic And Diagnostic Agents,” J. Biotechnol. 74:5-13 (2000).

### Anti-Sense Molecules

Another class of ubiquitin ligase inhibitors useful in certain embodiments is isolated antisense nucleic acid molecules that can hybridize to, or are complementary to, the nucleic acid molecule comprising a ubiquitin ligase nucleotide sequence, or fragments, analogs or derivatives thereof. An “antisense” nucleic acid comprises a nucleotide sequence that is complementary to a “sense” nucleic acid encoding a protein (e.g., complementary to the coding strand of a double-stranded cDNA molecule or complementary to an mRNA sequence). (See, for example, Uhlmann, et al. Antisense oligonucleotides: A new therapeutic principle. Chemical Reviews 1990, 90: 543-584; Crooke, et al. “Antisense Research and Applications”, CRC Press (1993); Mesmaekar, et al. “Antisense oligonucleotides,”, Acc. Chem. Res. 1995, 28: 366-374; Stein. “The experimental use of antisense oligonucleotides: a guide for the perplexed.” J. Clin. Invest. 2001, 108, 641-644, and U.S. Pat. Nos. 6,117,992; 6,127,121; 6,235,887; 6,232,463; 6,579,704; 5,596,091; 6,031,086 and 6,117,992, the disclosures of which are incorporated herein by reference in their entireties). In certain embodiments, antisense nucleic acid molecules may comprise a sequence complementary to at least about 10, 25, 50, 100, 250 or 500 nucleotides or an entire ubiquitin ligase coding strand, or to only a portion thereof. Nucleic acid molecules encoding fragments, homologs, derivatives and analogs of ubiquitin ligase antisense nucleic acids complementary to a ubiquitin ligase nucleic acid sequence may be used.

In one embodiment, an antisense nucleic acid molecule is antisense to a “coding region” of the coding strand of a nucleotide sequence encoding a ubiquitin ligase protein. The term “coding region” refers to the region of the nucleotide sequence comprising codons that are translated into amino acid residues. In another embodiment, the antisense nucleic acid molecule is antisense to a “conceding region” of the coding strand of a nucleotide sequence encoding the target protein. The term “conceding region” refers to 5′ and 3′ sequences that flank the coding region and that are not translated into amino acids (i.e., also referred to as 5′ and 3′ untranslated regions).

The antisense nucleic acid molecule can be complementary to the entire coding region of a ubiquitin ligase mRNA, but more preferably is an oligonucleotide that is antisense to only a portion of the coding or noncoding region of a ubiquitin ligase mRNA. For example, the antisense oligonucleotide can be complementary to the region surrounding the translation start site of a ubiquitin ligase mRNA. An antisense oligonucleotide can be, for example, about 5, 10, 15, 20, 25, 30, 35, 40, 45, or 50 nucleotides in length. An antisense nucleic acid of the invention can be constructed using chemical synthesis or enzymatic ligation reactions using procedures known in the art. For example, an antisense nucleic acid (e.g., an antisense oligonucleotide) can be chemically synthesized using naturally-occurring nucleotides or variously modified nucleotides designed to increase the biological stability of the molecules or to increase the physical stability of the duplex formed between the antisense and sense nucleic acids (e.g., phosphorothioate derivatives and acridine substituted nucleotides can be used).

Examples of modified nucleotides that can be used to generate the antisense nucleic acid include, without limitatio: 5-fluorouracil, 5-bromouracil, 5-chlorouracil, 5-iodouracil, hypoxanthine, xanthine, 4-acetylcytosine, 5-(carboxyhydroxylmethyl)uracil, 5-carboxymethylaminomethyl-2-thiouridin-e, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiour-acil, beta-D-mannosylqueosine, 5′-methoxycarboxymethyluracil, 5-methoxyuracil, 2-methylthio-N-6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl)uracil, (acp3)w, and 2,6-diaminopurine. Alternatively, the antisense nucleic acid can be produced biologically using an expression vector into which a nucleic acid has been subcloned in an antisense orientation (i.e., RNA transcribed from the inserted nucleic acid will be of an antisense orientation to a target nucleic acid of interest).

In certain embodiments, antisense nucleic acid molecules may be administered to a subject or generated in situ such that they hybridize with or bind to cellular mRNA and/or genomic DNA encoding a ubiquitin ligase to thereby inhibit expression of the protein (e.g., by inhibiting transcription and/or translation). The hybridization can be by conventional nucleotide complementarity to form a stable duplex, or, for example, in the case of an antisense nucleic acid molecule that binds to DNA duplexes, through specific interactions in the major groove of the double helix. An example of a route of administration of antisense nucleic acid molecules of the invention includes direct injection at a tissue site. Alternatively, antisense nucleic acid molecules can be modified to target selected cells and then administered systemically. For example, for systemic administration, antisense molecules can be modified such that they specifically bind to receptors or antigens expressed on a selected cell surface (e.g., by linking the antisense nucleic acid molecules to peptides or antibodies that bind to cell surface receptors or antigens).

In yet another embodiment, the antisense nucleic acid molecule of the invention is an alpha-anomeric nucleic acid molecule. An alpha-anomeric nucleic acid molecule forms specific double-stranded hybrids with complementary RNA in which, contrary to the usual alpha-units, the strands run parallel to each other. See, e.g., Gaultier, et al., Nucl. Acids Res., 15:6625-6641 (1987). The antisense nucleic acid molecule can also comprise a 2′-o-methylribonucleotide (see, e.g., Inoue, et al. Nucl. Acids Res., 15:6131-6148 (1987)) or a chimeric RNA-DNA analogue (see, e.g., Inoue, et al., FEBS Lett., 215:327-330 (1987)).

Production and delivery of antisense molecules may be facilitated by providing a vector comprising an anti-sense nucleotide sequence complementary to at least a part of the ubiquitin ligase DNA sequence. According to a yet further aspect of the invention such a vector comprising an anti-sense sequence may be used to inhibit ubiquitin ligase expression.

### RNA Interference

In certain embodiments, use of RNA interference to inactivate or modulate expression of a ubiquitin ligase is provided. RNA interference is described in U.S. Patent Application Publication No. 2002-0162126, and Hannon, G., J. Nature, 11:418:244-51 (2002).

RNAi is a process of sequence-specific post-transcriptional gene repression which can occur in eukaryotic cells. In general, this process involves degradation of an mRNA of a particular sequence induced by double-stranded RNA (dsRNA) that is homologous to that sequence. For example, the expression of a long dsRNA corresponding to the sequence of a particular single-stranded mRNA (ss mRNA) will labilize that message, thereby “interfering” with expression of the corresponding gene. Accordingly, any selected gene may be repressed by introducing a dsRNA which corresponds to all or a substantial part of the mRNA for that gene.

Mammalian cells have at least two pathways that are affected by double-stranded RNA (dsRNA). In the RNAi (sequence-specific) pathway, the initiating dsRNA is first broken into short interfering (si) RNAs. The siRNAs have sense and antisense strands of about 21 nucleotides that form approximately 19 nucleotide siRNAs with overhangs of two nucleotides at each 3′ end. Short interfering RNAs provide the sequence information that allows a specific messenger RNA to be targeted for degradation. In contrast, the nonspecific pathway is triggered by dsRNA of any sequence, as long as it is at least about 30 base pairs in length. Longer dsRNAs appear to be required to induce the nonspecific pathway and, accordingly, dsRNAs shorter than about 30 bases pairs are preferred to effect gene repression by RNAi (see Hunter et al. (1975) J. Biol. Chem. 250: 409-17; Manche et al. (1992) Mol. Cell Biol. 12: 5239-48; Minks et al. (1979) J. Biol. Chem. 254: 10180-3; and Elbashir et al. (2001) Nature 411: 494-8).

The double stranded oligonucleotides used to affect RNAi are preferably less than 30 base pairs in length and, more preferably, comprise about 25, 24, 23, 22, 21, 20, 19, 18 or 17 base pairs of ribonucleic acid. Optionally the dsRNA oligonucleotides may include 3′ overhang ends. Exemplary 2-nucleotide 3′ overhangs may be composed of ribonucleotide residues of any type and may even be composed of 2′-deoxythymidine resides. Longer dsRNAs of 50, 75, 100 or even 500 base pairs or more may also be utilized in certain embodiments of the invention. Exemplary concentrations of dsRNAs for effecting RNAi are about 0.05 nM, 0.1 nM, 0.5 nM, 1.0 nM, 1.5 nM, 25 nM or 100 nM, although other concentrations may be utilized depending upon the nature of the cells treated, the gene target and other factors readily discernable to the skilled artisan. The dsRNAs may be synthesized chemically or produced in vitro or in vivo using appropriate expression vectors., or may be obtained commercially Exemplary synthetic RNAs include 21 nucleotide RNAs chemically synthesized using methods known in the art. Synthetic oligonucleotides are preferably deprotected and gel-purified using methods known in the art (see e.g. Elbashir et al. (2001) Genes Dev. 15: 188-200). Longer RNAs may be transcribed from promoters, such as T7 RNA polymerase promoters, known in the art. A single RNA target, placed in both possible orientations downstream of an in vitro promoter, will transcribe both strands of the target to create a dsRNA oligonucleotide of the desired target sequence. Any of the above RNA species may be designed to include a portion of nucleic acid sequence represented in a ubiquitin ligase nucleic acid.

The dsRNA need only be sufficiently similar to the target mRNA such that it mediates RNAi. The dsRNA may have at least 50%, preferably at least 70%, more preferably at least 80%, and most preferably at least 90% sequence identity with the target.

A preferred interfering RNA of the present invention is a siRNA, particularly small hairpin RNA (shRNA). siRNA, particularly shRNA, mediate the post-transcriptional process of gene silencing by double stranded RNA (dsRNA) that is homologous in sequence to the silenced RNA. siRNA according to the present invention preferably comprises a sense strand of 15-30 nucleotides, and an antisense strand of 15-30 nucleotides complementary to the sense strand. The siRNA preferably further comprises a loop region linking the sense and the antisense strand.

The siRNAs may be modified by methods known in the art for example by modified internucleoside linkages, modified nucleic acid bases, modified sugars and/or chemical linkage the siRNA to one or more moieties or conjugates.

The specific sequence utilized in design of the oligonucleotides may be any contiguous sequence of nucleotides contained within the expressed gene message of ubiquitin ligase. Programs and algorithms known in the art may be used to select appropriate target sequences. In addition, optimal sequences may be selected utilizing programs designed to predict the secondary structure of a specified single stranded nucleic acid sequence and allowing selection of those sequences likely to occur in exposed single stranded regions of a folded mRNA. Methods and compositions for designing appropriate oligonucleotides may be found, for example, in U.S. Pat. No. 6,251,588.

The RNA oligonucleotides may be introduced into a cell by methods known in the art for introducing ribonucleic acids into animal cells and disclosed for example in U.S. Patent Application Publication Nos. 20100120891 and 20110065908. For example, transfection with using carrier compositions such as liposomes, are known in the art—e.g. Lipofectamine 2000 (Life Technologies). Transfection of dsRNA oligonucleotides for targeting endogenous genes may be carried out using Oligofectamine (Life Technologies). Nanoparticles such as the cationic polymer polyethyleneimine (PEI) may also be used to deliver siRNA to target cells. The RNA oligonucleotides may also be delivered by viral transduction utilizing, for example, an adenoviral, lentiviral, baculoviral, or adeno-associated viral vector. Other nonlimiting examples include the use of modified viral particles and implantable drug-releasing biodegradable microspheres.

Further compositions, methods and applications of RNAi technology are provided in U.S. Pat. Nos. 6,278,039, 5,723,750 and 5,244,805.

### Formulations

Formulations of the compositions useful in certain embodiments such as polypeptides, polynucleotides, or antibodies may be prepared for storage by mixing the selected composition having the desired degree of purity with optional physiologically pharmaceutically-acceptable carriers, excipients, or stabilizers (Remington's Pharmaceutical Sciences, 18th edition, A. R. Gennaro, ed., Mack Publishing Company (1990)) in the form of a lyophilized cake or an aqueous solution. Acceptable carriers, excipients or stabilizers are nontoxic to recipients and are preferably inert at the dosages and concentrations employed, and include buffers such as phosphate, citrate, or other organic acids; antioxidants such as ascorbic acid; low molecular weight polypeptides; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as Tween, Pluronics or polyethylene glycol (PEG).

Compositions to be used for in vivo administration should be sterile. This is readily accomplished by filtration through sterile filtration membranes, prior to or following lyophilization and reconstitution. The composition for parenteral administration ordinarily will be stored in lyophilized form or in solution.

Therapeutic compositions generally are placed into a container having a sterile access port, for example, an intravenous solution bag or vial having a stopper pierceable by a hypodermic injection needle. The route of administration of the composition is in accord with known methods, e.g. oral, injection or infusion by intravenous, intraperitoneal, intracerebral, intramuscular, intraocular, intraarterial, or intralesional routes, or by sustained release systems or implantation device. Where desired, the compositions may be administered continuously by infusion, bolus injection or by implantation device.

An effective amount of the compositions to be employed therapeutically will depend, for example, upon the therapeutic objectives, the route of administration, and the condition of the patient. Accordingly, it may be necessary for the therapist to titer the dosage and modify the route of administration as required to obtain the optimal therapeutic effect. A typical daily dosage may range from about 1 μg/kg to up to 100 mg/kg or more, depending on the factors mentioned above. Typically, a clinician will administer the composition until a dosage is reached that achieves the desired effect. The progress of this therapy is easily monitored by conventional assays designed to evaluate blood glucose levels or other particular conditions of interest in a particular subject.

Pharmaceutical compositions may be produced by admixing a pharmaceutically effective amount of protein with one or more suitable carriers or adjuvants such as water, mineral oil, polyethylene glycol, starch, talcum, lactose, thickeners, stabilizers, suspending agents, etc. Such compositions may be in the form of solutions, suspensions, tablets, capsules, creams, salves, ointments, or other conventional forms.

In certain embodiments, compounds are formulated with pharmaceutically acceptable diluents, adjuvants, excipients, or carriers. The phrase “pharmaceutically or pharmacologically acceptable” refers to molecular entities and compositions that do not produce adverse, allergic, or other untoward reactions when administered to an animal or a human, e.g., orally, topically, transdermally, parenterally, by inhalation spray, vaginally, rectally, or by intracranial injection. The term parenteral as used herein includes subcutaneous injections, intravenous, intramuscular, intracistemal injection, or infusion techniques. Administration by intravenous, intradermal, intramusclar, intramammary, intraperitoneal, intrathecal, retrobulbar, intrapulmonary injection and/or surgical implantation at a particular site is contemplated as well.) Generally, this will also entail preparing compositions that are essentially free of pyrogens, as well as other impurities that could be harmful to humans or animals. The term “pharmaceutically acceptable carrier” includes any and all solvents, dispersion media, coatings, antibacterial and antifungal agents, isotonic and absorption delaying agents, liposomes, capsids, nanocapsules, microcapsules and the like. The use of such media and agents for pharmaceutically active substances is well known in the art.

The pharmaceutical forms suitable for injectable use include sterile aqueous solutions or dispersions and sterile powders for the extemporaneous preparation of sterile injectable solutions or dispersions. The carrier can be a solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion and by the use of surfactants. The prevention of the action of microorganisms can be brought about by various antibacterial an antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

### Routes of Administration

In certain embodiments, the present invention provides a method of treating a subject comprising administration of a composition. As used herein, the term “subject” is used to mean an animal, preferably a mammal, including a human. The terms “patient” and “subject” may be used interchangeably.

The therapeutic compositions may be administered by any route that delivers an effective dosage to the desired site of action, with acceptable (preferably minimal) side-effects. Numerous routes of administration of agents are known, for example, oral, rectal, transmucosal, or intestinal administration; parenteral delivery, including intramuscular, subcutaneous, intramedullary injections, as well as intrathecal, intraperitoneal, intranasal, cutaneous or intradermal injections; inhalation, and topical application.

Therapeutic dosing is achieved by monitoring therapeutic benefit and monitoring to avoid side-effects. Preferred dosage provides a maximum localized therapeutic benefit with minimum local or systemic side-effects. Suitable human dosage ranges for the polynucleotides or polypeptides can be extrapolated from these dosages or from similar studies in appropriate animal models. Dosages can then be adjusted as necessary by the clinician to provide maximal therapeutic benefit for human subjects.

When a therapeutically effective amount of a composition of the present invention is administered by e.g., intradermal, cutaneous or subcutaneous injection, the composition is preferably in the form of a pyrogen-free, parenterally acceptable aqueous solution. The preparation of such parenterally acceptable protein or polynucleotide solutions, having due regard to pH, isotonicity, stability, and the like, is within the skill in the art. A preferred composition should contain, in addition to protein or other active ingredient of the present invention, an isotonic vehicle such as Sodium Chloride Injection, Ringer's Injection, Dextrose Injection, Dextrose and Sodium Chloride Injection, Lactated Ringer's Injection, or other vehicle as known in the art. The composition of the present invention may also contain stabilizers, preservatives, buffers, antioxidants, or other additives known to those of skill in the art. The agents of the invention may be formulated in aqueous solutions, preferably in physiologically compatible buffers such as Hanks's solution, Ringer's solution, or physiological saline buffer. For transmucosal administration, penetrants appropriate to the barrier to be permeated are used in the formulation. Such penetrants are generally known in the art.

The compositions of the invention may be in the form of a complex of the protein(s) or other active ingredient of present invention along with protein or peptide antigens.

The composition may further contain other agents which either enhance the activity of the protein or other active ingredient or complement its activity or use in treatment. Such additional factors and/or agents may be included in the pharmaceutical composition to produce a synergistic effect with protein or other active ingredient, or to minimize side effects.

Techniques for formulation and administration of the therapeutic compositions of the instant application may be found in “Remington's Pharmaceutical Sciences,” Mack Publishing Co., Easton, Pa., latest edition. When applied to an individual active ingredient, administered alone, a therapeutically effective dose refers to that ingredient alone. When applied to a combination, a therapeutically effective dose refers to combined amounts of the active ingredients that result in the therapeutic effect, whether administered in combination, serially or simultaneously.

The following examples serve to further illustrate the present invention.

### EXAMPLE 1

### Materials and Methods

### Cells and Cell Culture

U87 MG and U118 MG (hereafter abbreviated as U87, U118 respectively) human glioblastoma (glioma) cell lines were kindly provided by Dr. Hui Zhang and Dr. Hong Sun. MCF7 (human breast cancer), H1299 (non-small cell lung carcinoma), and PC3 (human prostate cancer) cell lines were obtained from Dr. Giuseppe Pizzorno. Normal human dermal fibroblasts, HDF and normal human mammary epithelial cells, HMEpC, were purchased from Cell Applications (San Diego, Calif.). U118, U87, and MCF7 cultures were grown in high-glucose DMEM supplemented with 10% Fetal Bovine Serum (FBS, HyClone, Waltham, Mass.) and 1% Penicillin/Streptomycin. H1299 was grown in RPMI with 10% FBS and 1% Pen/Strep. PC3 cells were in F12K Medium with 10% FBS and 1% antibiotics. HDF was grown in Human Fibroblast Growth Medium (Cell Applications, San Diego, Calif.) and HMEpC in Mammary Epithelial Growth Medium (Cell Applications). All cells were grown at 37° C. in a humidified atmosphere of 95% air and 5% CO₂. Cell number was measured by a hemacytometer (Hausser Scientific, Horsham, Pa.). Cell population doubling time (T_{d}) was calculated from the exponential portion of the growth curve using the equation T_{d}=0.693t/ln(Nₜ/N₀), where t is time, and Nₜ and N₀ represent cell numbers at time t and initial time, respectively (Y. Zhang, et al., Cancer Res 62 (2002) 1205-12).

### Resveratrol Treatment

For short-term resveratrol treatment, cells in 60 mm dishes were allowed to grow to 40%-50% confluency. Their original media were changed to 5 ml DMEM media containing glucose (4.5 g/L) as the only carbon source (without both L-glutamine and sodium pyruvate). After one hour of media change, cells were collected as non-treated or treated with 5 μL acetone or resveratrol in 5 μL acetone vehicle for 4 hours and then collected for Western analysis. For long-term resveratrol treatment, 3×10⁵ of U87 or U118 glioma cells were plated in 100 mm dishes. One day later, the culture media were replaced with fresh medium (10 mL/dish). After one more day, the cells were mock-treated, or 10 μL vehicle acetone, or treated with 6 μM, 10 μM, and 20 μM resveratrol (final concentration in medium) for U118 cells, or 6 μM, 20 μM, and 60 μM resveratrol (final concentration in medium) for U87 cells. The media (with or without resveratrol) were changed every 3 days until collection.

### Western Analysis

Cultured cells were harvested on ice (3-5 min) by scraping in the medium, centrifuged with about 300 relative centrifugal forces (RCF) for 2 minutes. The cell pellets were then re-suspended in 2×SDS-PAGE sample buffer containing 2-mercaptoethanol and heated at 100° C. for 5 minutes, and stored at −80° C. until use. Total protein was separated by 12% SDS polyacrylamide gel electrophoresis and analyzed with antibodies to H2B (Millipore, Billerica, Mass.), mono-ubiquitinated H2B (uH2B, MediMabs, Montreal, Quebec, Canada), RNF20 (Abcam, Cambridge, Mass.), p53 (Santa Cruz Biotechnology, Santa Cruz, Calif.). Immunoreactive bands were detected using SuperSignal kit (Thermo Scientific, Waltham, Mass.).

Senescence Associated-β-galactosidase (SA-β-Gal) Staining

Senescence-β-galactosidase staining was performed with a kit (Cat. #9860, Cell Signaling, Danvers, Mass.) according to manufacturer's protocol. Briefly, cells in 35 mm well were washed with 2 mL PBS, fixed for 10-15 minutes in 2 mL fixative solution, washed twice with 2 mL PBS, and then incubated in SA-β-gal staining solution overnight at 37° C.

### Clonogenic Assays

Monolayer cultures of glioma cells were trypsinized and appropriate cell dilutions were re-plated. After 14 days, cultured cells were stained with 0.4% Coomassie Blue G250 in 50% methanol and 5% acetic acid. Colonies of 50 cells or more were counted. Plating efficiencies (PE) were calculated using the formula PE=(number of colonies counted/number of cells seeded)×100%.

### Growth of Human Tumor Xenograft

U87 or U118 Cells (2,000,000 in 0.1 ml PBS) were injected subcutaneously in the right flank of the female nude (nu/nu) mice (4-6 weeks old; Charles River Laboratories, Wilmington, Mass.). Five nude mice were used for each group. U87 tumor volume was measured at 27 days post injection while U118 tumor volume at 125 days post injection. When tumors were palpable, they were measured by a Vernier caliper in indicated days after injection. Tumor volume (TV) was calculated as follows: TV (mm³)=LXW²/2, where L is the longest dimension of the tumor (in mm), and W is the shortest dimension of the tumor.

Lentiviral shRNA Knockdown Experiments

Lentiviral particles for knockdown of RNF20 (TRCN 0000033877, hereafter is abbreviated as shRNF20) were purchased from Sigma-Aldrich (Mission collection, St. Louis, Mo.) and contain the inserted sequence:

(SEQ I.D.No: 1)
CCGGGCCAATGAAATCAAGTCTAAACTCGAGTTTAGACTTGATTTCATTG
GCTTT TG.

Control lentiviral particles are Luciferase (SHC007, hereafter abbreviated as shLuc) were from Sigma-Aldrich containing the sequence:

(SEQ. I.D. No: 2)
CCGGCGCTGAGTACTTCGAAATGTCTCGA GGACATTT
CGAAGTACTCAGCGTTTTT.

Lentiviral transduction was performed according to the manufacturer's instructions. Briefly, U87 was seeded at 1.5×10⁵ cells per 100 mm dishes while U118 was seeded at 3.0×10⁵ cells per 100 mm dishes. Two days later, hexadimethrine bromide (Sigma-Aldrich) was added to medium (final concentration 8 μg/mL) to enhance transduction efficiency. Then shRNF20 or shLuc {multiplicity of infection (MOI)=10} was added and the plates were swirled gently for mixing. The viral particle-containing medium was replaced with fresh medium next day. After one more day, the media was replaced with fresh medium containing 2 μg/mL puromycin (Sigma-Aldrich) for selecting transfected cells. The puromycin containing media were then changed every 3 days.

### Statistical Analysis

Tumor xenograft was performed with 5 mice in each group; Western experiments were repeated at least twice or more; all other experiments were performed at least three times. Data were analyzed by R Integrated Statistical Software (version 2.6.0, Vienna, Austria). Student t test was used to evaluate the significance of difference. p<0.05 was accepted as significant.

### EXAMPLE 2

### Resveratrol Inhibited Cell Proliferation and Induced Cellular Senescence in Cultured Glioma Cells

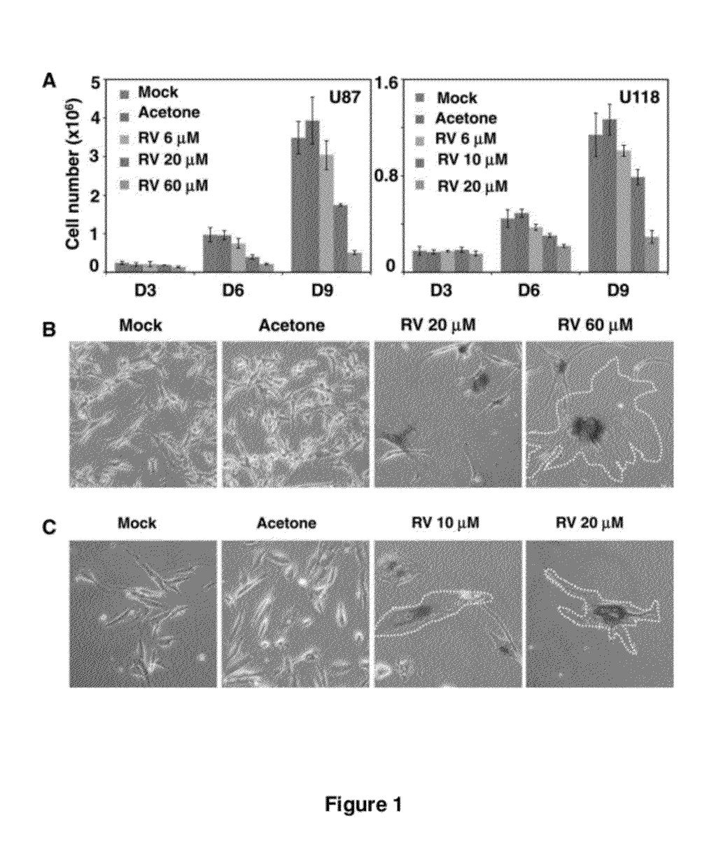

To examine the effects of resveratrol on glioma cells, 5×10⁴ U87 or U118 glioblastoma (Grade IV glioma cell) lines were plated in 60 mm dishes. The cells were treated with mock, acetone, or different doses of resveratrol that were dissolved in acetone. Cells were then trypsinized and cell numbers counted at 3, 6, and 9 days post treatment. As shown in FIG. 1A, resveratrol treatment significantly inhibited proliferation of these two human glioma cell lines on a dose- and time-dependent manner.

Closer examination of glioma cells indicated that resveratrol treatment inhibited cell proliferation by inducing cellular hypertrophy in glioma cells. Resveratrol induced dramatic changes in cell volume and cell morphology in both U87 and U118 cells. Specifically, resveratrol transformed spindle-shaped glioma cells to enlarged and irregular flatten-shaped ones. The mean diameter of the resveratrol-treated U87 was 5 fold longer than the diameter parallel to the spindle axis of the mock-or vehicle-treated glioma cells. In contrast, mock and vehicle (acetone) treatment had no effect on inducing cellular hypertrophy.

To determine whether cellular hypertrophy induced by resveratrol was associated with cellular senescence of glioma cells, expression of senescence-associated-β-galactosidase (SA-β-gal) marker was tested (G. P. Dimri, et al., Proc Natl Acad Sci USA 92 (1995) 9363-7; G. P. Dimri, et al., Biol Signals 5 (1996) 154-62). As shown FIG. 1B-C, SA-β-gal was detected in these hypertrophic cells. In contrast, mock- or vehicle-treated cells showed minimal expression of SA-β-gal even at a higher density. These results demonstrated that resveratrol inhibited proliferation of the glioma cells by inducing cellular senescence.

### EXAMPLE 3

### Resveratrol Treatment Reduced Tumorigencity of Glioma Cells

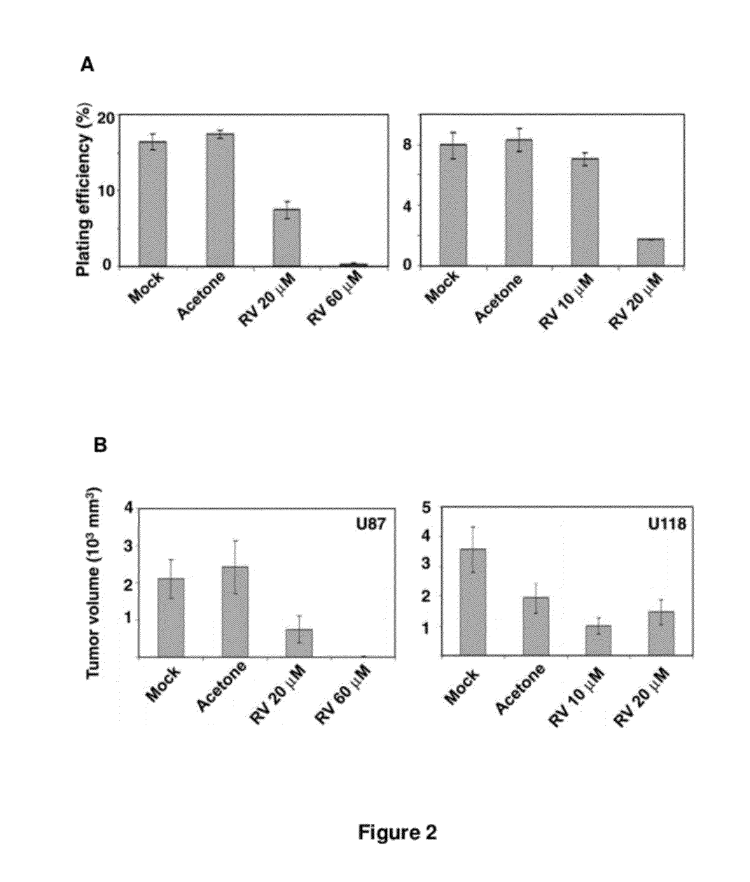

To determine whether resveratrol-induced senescent cells had an impact on clonogenicity, U87 and U118 cells were treated with the indicated concentrations of resveratrol for up to 9 days. Monolayer cultures of glioma cells were trypsinized and seeded 1000, 2000, and 4000 cells separately in 60 mm culture dishes for incubation without further resveratrol treatment. After two weeks, these cell cultures were stained with 0.4% Coomassie Blue G250 in 50% methanol and 5% acetic acid. Colonies that contained more than 50 cells were counted. Cell clonogenicity was calculated with plating efficiencies (PE) using the formula PE=(number of colonies counted/number of cells seeded)×100%. As shown in FIG. 2A, resveratrol-treated cells showed marked decrease in their propensity for forming colonies in a dose-dependent manner.

To further determine if resveratrol treatment reduced in vivo tumorigenicity of U87 and U118 glioma cells, the cells were treated with the indicated concentrations of resveratrol for 9 days. Glioma cells were then trypsinized, and 2×10⁶ viable cells (by Trypan blue exclusion assay) were collected in 0.1 ml PBS. These cells were subsequently injected subcutaneously in the right flank of nude mice [female (nu/nu), 4-6 weeks old, 5 mice per group]. When the tumor was palpable, the size was measured with a Vernier caliper. Tumor volume was calculated (TV) using the formula: TV (mm³)=LXW²/2, in which L was the longest dimension of the tumor (in mm), and W was the shortest dimension of the tumor. As shown in FIG. 2B, resveratrol inhibited the tumorigenecity of both U87 and U118 in the xenograft model.

### EXAMPLE 4

### Resveratrol Inhibited Mono-Ubiquitination of Histone H2B (uH2B) at K120 in Glioma Cells

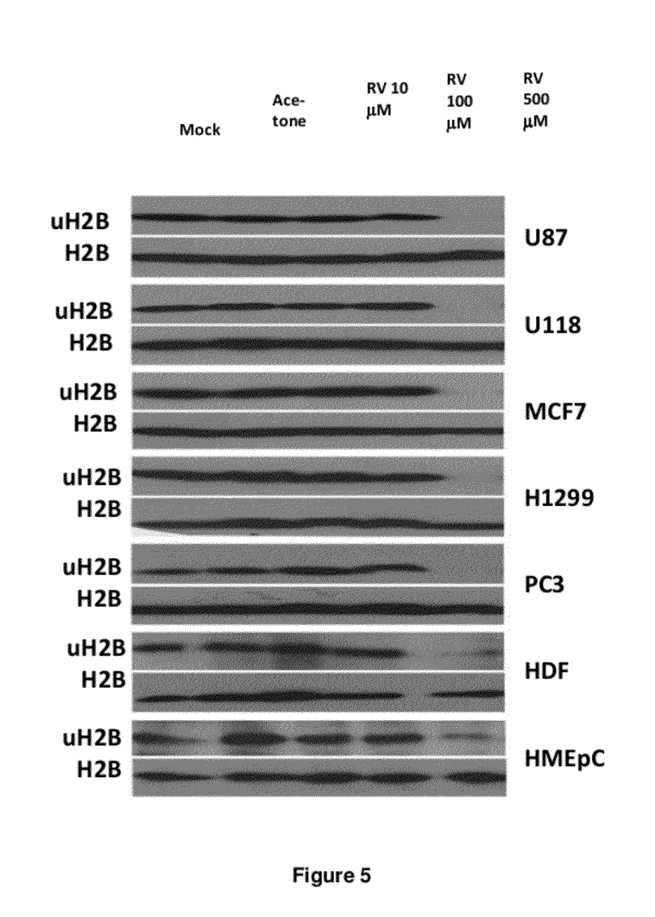

The effect of resveratrol treatment on uH2B at K120 in human cells, which include glioma cells (U87, U118), breast cancer (MCF7), non-small lung carcinoma cells (H1299), human prostate cells (PC3), non-malignant normal human dermal fibroblast and human mammary epithelial cells (HMEpC) was assessed. Cells were grown to around 50% confluency in regular DMEM media (2 to 3 days) and media was replaced with DMEM media (with glucose 4.5 g/L, but without both glutamine and sodium pyruvate) for 1 hour. Resveratrol was added (10, 100, 500 μM) for 4 h and subsequently cells were collected for Western analysis of uH2B. As shown in FIG. 5, resveratrol inhibited uH2B within 4 hours in a dose-dependent manner.

Since acute incubation with the high dose of resveratrol (500 μM) was effective in inhibiting uH2B, it was then examined whether chronic treatment with lower doses of resveratrol had any effect on uH2B. To test this, glioma cells (U87 and U118) were employed. As shown in FIG. 3A and B, resveratrol inhibited uH2B of U87 and U118 in a dose-dependent manner. Resveratrol also inhibited uH2B of U87 and U118 in a dose-dependent manner. These data indicate that resveratrol inhibits mono-ubquitination of histone H2B at K120 in glioma cells.

### EXAMPLE 5

### Depletion of RNF20, an Ubiquitin Ligase of Histone H2B, Phenocopied the Effects of Resveratrol by Inhibiting uH2B and Inducing Cellular Senescence in Human Glioma Cells

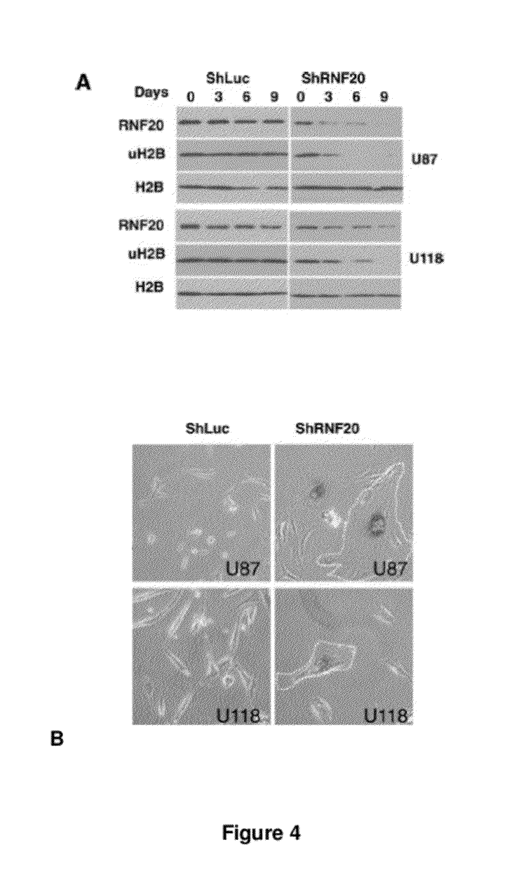

Since resveratrol inhibited uH2B and induced cellular senescence, it was investigated whether depletion of RNF20, a ubiquitin ligase of histone H2B, could mimic the effects of resveratrol. To test this possibility, lentiviral particles (Sigma, St Louis, Mo.) expressing short hairpin RNA (shRNA) of RNF20 (referred as shRNF20 thereafter) were used to knockdown RNF20 in U87 and U118 glioma cells. Lentiviral particles encoding shRNA for luciferase (referred as shLuc thereafter) were used as a control. As shown in FIG. 4A, treatment with shRNF20 significantly reduced expression of RNF20 and almost completely abolished uH2B in a time-dependent manner. In contrast, shLuc treatment had no effect on either RNF20 or uH2B levels. These data demonstrate that that RNF20 is the major ubiquitin ligase for histone H2B.

It was then examined whether depletion of RNF20 could induce cellular senescence in glioma cells. Depletion of RNF20 induced similar cellular hypertrophy as observed with resveratrol treatment. Specifically, depletion of RNF20 transformed spindle-shaped glioma cells into extremely enlarged and irregular flatten-shaped ones (data not shown). As shown in FIG. 4B, the hypertrophic cells expressed a high level of SA-β-gal. These data indicate that depletion of RNF20 recapitulates the inhibitory effects of resveratrol treatment of glioma cells.

All references cited herein are incorporated by reference herein in their entireties.

We claim:

1. A method of inducing senescence in a cell comprising contacting the cell with an inhibitor of mono-ubiquitination of a histone.

2. The method of claim 1 wherein the cell is a cancer cell.

3. The method of claim 1 wherein the cell is a glioma cell.

4. The method of claim 1 wherein the histone is histone H2B.

5. The method of claim 1 wherein the inhibitor of mono-ubiquitination of the histone comprises a negative modulator of a ubiquitin ligase.

6. The method of claim 5 wherein the ubiquitin ligase is RNF20.

7. The method of claim 5 wherein the negative modulator of the ubiquitin ligase is selected from the group consisting of: an anti-ubiquitin ligase antibody, an aptamer that inhibits ubiquitin ligase, an antisense nucleic acid molecule that inhibits ubiquitin ligase transcription or translation, an siRNA that inhibits ubiquitin ligase translation, a small molecule inhibitor of ubiquitin ligase, and combinations thereof.

8. The method of claim 7 wherein the small molecule inhibitor is resveratrol.

9. A method of inhibiting mono-ubiquitination of a histone in a cell comprising contacting the cell with a negative modulator of a ubiquitin ligase.

10. The method of claim 9 wherein the cell is selected from the group consisting of breast, prostate, pancreatic, lung, and brain tumor cells.

11. The method of claim 9 wherein the histone is histone H2B.

12. The method of claim 9 wherein the ubiquitin ligase is RNF20.

13. The method of claim 9 wherein the negative modulator of the ubiquitin ligase is selected from the group consisting of: an anti-ubiquitin ligase antibody, an aptamer that inhibits ubiquitin ligase, an antisense nucleic acid molecule that inhibits ubiquitin ligase transcription or translation, an siRNA that inhibits ubiquitin ligase translation, a small molecule inhibitor of ubiquitin ligase, and combinations thereof.

14. The method of claim 13 wherein the small molecule inhibitor is resveratrol.

15. A method of treatment for cancer comprising administration to a subject in need of treatment a composition comprising a negative modulator of a ubiquitin ligase in an amount effective to inhibit mono-ubiquitination of a histone in cancer cells in the subject.

16. The method of claim 15 wherein the cancer is glioma.

17. The method of claim 15 wherein the histone is histone H2B.

18. The method of claim 15 wherein the ubiquitin ligase is RNF20.

19. The method of claim 15 wherein the negative modulator of the ubiquitin ligase is selected from the group consisting of: an anti-ubiquitin ligase antibody, an aptamer that inhibits ubiquitin ligase, an antisense nucleic acid molecule that inhibits ubiquitin ligase transcription or translation, an siRNA that inhibits ubiquitin ligase translation, a small molecule inhibitor of ubiquitin ligase, and combinations thereof.

20. The method of claim 19 wherein the small molecule inhibitor is resveratrol.

21. A composition effective for the treatment of cancer comprising at least one negative modulator of a ubiquitin ligase.

22. The composition of claim 21 wherein the negative modulator of the ubiquitin ligase is selected from the group consisting of: an anti-ubiquitin ligase antibody, an aptamer that inhibits ubiquitin ligase, an antisense nucleic acid molecule that inhibits ubiquitin ligase transcription or translation, an siRNA that inhibits ubiquitin ligase translation, a small molecule inhibitor of ubiquitin ligase, and combinations thereof.

23. The composition of claim 22 wherein the small molecule inhibitor is resveratrol.


SEQUENCE LISTING (2 sequences)

SEQ ID NO: 1 - DNA, 58 bp, Artificial
  Synthetic lentiviral particle
ccgggccaatgaaatcaagtctaaactcgagtttagacttgatttcattggctttttg

SEQ ID NO: 2 - DNA, 56 bp, Artificial
  Synthetic lentiviral particle
ccggcgctgagtacttcgaaatgtctcgaggacatttcgaagtactcagcgttttt